(19) United States
(12) Patent Application Publication
(10) Pub. No.: US 2024/0245449 A1
(43) Pub. Date: Jul. 25, 2024

(54) RF ABLATION SYSTEMS WITH AT LEAST ONE DIRECTIONAL ELEMENT AND METHODS FOR MAKING AND USING

(71) Applicant: Boston Scientific Neuromodulation Corporation, Valencia, CA (US)

(72) Inventor: Gregory Bates, Bend, OR (US)

(21) Appl. No.: 18/414,919

(22) Filed: Jan. 17, 2024

Related U.S. Application Data
(60) Provisional application No. 63/440,616, filed on Jan. 23, 2023.

(51) Int. Cl. A61B 18/14

(52) U.S. Cl. CPC A61B 18/1482; A61B 2018/00083

(57) ABSTRACT

A bipolar RF electrode or other RF electrode can include one or more directional elements, such as one or more directional electrodes or directional fluid flow portions. For example, a bipolar RF electrode can include an electrode shaft having a circumference, a first end portion, and a second end portion opposite the first end portion; a first electrode coupled to the second end portion of the electrode shaft, wherein the first electrode extends around no more than 80% of the circumference of the electrode shaft; a second electrode coupled to the second end portion of the electrode shaft; an insulative material coupled to, and disposed between, the first electrode and the second electrode; and an electrode hub attached to the first end portion of the electrode shaft.

### CROSS-REFERENCE TO RELATED APPLICATIONS

This application claims the benefit under 35 U.S.C. § 119(e) of U.S. Provisional Patent Application Ser. No. 63/440,616, filed Jan. 23, 2023, which is incorporated herein by reference.

### FIELD

The present disclosure is directed to the area of radiofrequency (RF) ablation systems and methods of making and using the systems. The present disclosure is also directed to RF ablation systems and methods that include at least one directional element, as well as methods of making and using the same.

### BACKGROUND

Radiofrequency (RF) generators and electrodes can be used for pain relief or functional modification. Radiofrequency ablation (RFA) is a safe, proven means of interrupting pain signals, such as those coming from irritated facet joints in the spine, genicular nerves in the knee, and femoral and obturator nerves in the hip. Radiofrequency current is used to heat up a small volume of nerve tissue, thereby interrupting pain signals from that specific area. Radiofrequency ablation is designed to provide long-lasting pain relief.

For example, an RF electrode can be positioned near target tissue and then used to heat the target tissue by RF power dissipation of the RF signal output in the target tissue. Temperature monitoring of the target tissue by a temperature sensor in the electrode may be used to control the process.

### BRIEF SUMMARY

One aspect is a bipolar RF electrode that includes an electrode shaft having a circumference, a first end portion, and a second end portion opposite the first end portion; a first electrode coupled to the second end portion of the electrode shaft, wherein the first electrode extends around no more than 80% of the circumference of the electrode shaft; a second electrode coupled to the second end portion of the electrode shaft; an insulative material coupled to, and disposed between, the first electrode and the second electrode; and an electrode hub attached to the first end portion of the electrode shaft.

In at least some aspects, the second electrode extends around no more than 80% of the circumference of the electrode shaft. In at least some aspects, both the first electrode and the second electrode extend around no more than 50% of the circumference of the electrode shaft. In at least some aspects, the first electrode and the second electrode are aligned on the circumference of the electrode shaft.

In at least some aspects, the insulative material defines at least one fluid delivery port. In at least some aspects, the electrode hub or the electrode shaft is configured for attachment of a fluid line, wherein at least the electrode shaft, the second electrode, and the insulative material form a hollow interior for flow of fluid from the electrode hub or the electrode shaft to the at least one fluid delivery port defined by the insulative material and disposed between the first electrode and the second electrode. In at least some aspects, the at least one fluid delivery port is a single fluid delivery port directionally aligned with the first electrode.

In at least some aspects, the bipolar RF electrode further includes a marker disposed on the electrode hub, within the electrode shaft, or beneath the first or second electrode to indicate circumferential orientation or position of the first electrode.

Another aspect is a bipolar RF electrode that includes an electrode shaft having a first end portion and a second end portion opposite the first end portion; a first electrode coupled to the second end portion of the electrode shaft; a second electrode coupled to the second end portion of the electrode shaft; an insulative material coupled to, and disposed between, the first electrode and the second electrode, the insulative material having a circumference and defining one or more fluid delivery ports, wherein, when the one or more fluid delivery ports is a single fluid delivery port, the single fluid delivery port extends around no more than 50% of the circumference of the insulative material and, when the one or more fluid delivery ports is at least two fluid delivery ports, all of the at least two fluid delivery ports are defined in a contiguous portion of the insulative material that extends around no more than 50% of the circumference of the insulative material; and an electrode hub attached to the first end portion of the electrode shaft, wherein the electrode hub or the electrode shaft is configured for attachment of a fluid line, wherein at least the electrode shaft, the second electrode, and the insulative material form a hollow interior for flow of fluid from the electrode hub or the electrode shaft to the at least one fluid delivery port defined by the insulative material and disposed between the first electrode and the second electrode.

In at least some aspects, the first electrode extends around no more than 80% of the circumference of the electrode shaft. In at least some aspects, both the first electrode and the second electrode extend around no more than 50% of the circumference of the electrode shaft.

In at least some aspects, the bipolar RF electrode further includes a marker disposed on the electrode hub, within the electrode shaft, or beneath the first or second electrode to indicate circumferential orientation or position of the at least one fluid delivery port.

A further aspect is a RF electrode that includes an electrode shaft having a circumference, a first end portion, and a second end portion opposite the first end portion; at least one electrode coupled to the second end portion of the electrode shaft, wherein the at least one electrode includes a first electrode that extends around no more than 80% of the circumference of the electrode shaft; and an electrode hub attached to the first end portion of the electrode shaft.

In at least some aspects, the first electrode extends around no more than 50% of the circumference of the electrode shaft. In at least some aspects, the electrode shaft defines at least one fluid delivery port. In at least some aspects, the electrode hub or the electrode shaft is configured for attachment of a fluid line, wherein at least the electrode shaft forms a hollow interior for flow of fluid from the electrode hub or electrode shaft to the at least one fluid delivery port defined by the electrode shaft. In at least some aspects, the at least one fluid delivery port is a single fluid delivery port directionally aligned with the first electrode.

In at least some aspects, the RF electrode further includes a marker disposed on the electrode hub, within the electrode shaft, or beneath the first or second electrode to indicate circumferential orientation or position of the first electrode.

Yet another aspect is a RF ablation system that includes any of the bipolar RF electrodes or RF electrodes described above; a cannula configured for insertion of the electrode shaft through the cannula; and a RF generator configured for electrically coupling to the RF electrode and energizing the at least one of the at least one electrode.

A further aspect is a kit that includes any of the bipolar RF electrodes or RF electrodes described above and a cannula configured for insertion of the electrode shaft through the cannula.

### BRIEF DESCRIPTION OF THE DRAWINGS

Non-limiting and non-exhaustive embodiments of the present invention are described with reference to the following drawings. In the drawings, like reference numerals refer to like parts throughout the various figures unless otherwise specified.

For a better understanding of the present invention, reference will be made to the following Detailed Description, which is to be read in association with the accompanying drawings, wherein:

FIG. 1 is a schematic side view of components of one embodiment of a RF ablation system with a bipolar RF electrode;

FIG. 2 is a schematic side view of components of one embodiment of an adapter for coupling a bipolar RF electrode to a RF generator;

FIG. 3 is a schematic perspective view of a distal portion of one embodiment of a bipolar RF electrode and cannula, where the bipolar RF electrode includes directional elements such as direction electrodes and at least one fluid delivery port;

FIG. 4 is a schematic cross-sectional view of a portion of the bipolar RF electrode of FIG. 3 illustrating energy flow from a directional electrode;

FIG. 5 is a schematic cross-sectional view of a portion of the bipolar RF electrode of FIG. 3 illustrating directional fluid flow from a fluid delivery port; and

FIG. 6 is a schematic diagram of another embodiment of a bipolar RF electrode and cannula with a fluid line and connector coupled to the bipolar RF electrode for delivery of fluid through the at least one fluid delivery port in the RF bipolar electrode.

### DETAILED DESCRIPTION

The present disclosure is directed to the area of radiofrequency (RF) ablation systems and methods of making and using the systems. The present disclosure is also directed to RF ablation systems and methods that include at least one directional element, as well as methods of making and using the same.

The basivertebral nerve (BVN) is located at the center of vertebrae in the lower back. The BVN can be difficult to access. To improve effectiveness, the ablation volume of the nerve should be large enough to eliminate the pain and prevent the nerve from growing back quickly. The location of the BVN can vary in vertebrae. Placement of the ablation electrode(s) may not provide full ablation due to the variation in the location of the BVN. This may decrease the therapy effectiveness. Furthermore, the patient may experience pain after the procedure due to trauma inside the vertebrae caused by the sharp access tools and the ablation itself.

The positioning of the ablation electrode(s) is often a tradeoff between generating an ablation volume that overlaps the BVN nerve root for efficacy and is sufficient distance away from spinal column to prevent harm to the patient by, for example, heating of the spinal fluid or nerves. Currently, the BVN ablation procedure is only approved in the United States for L3-S1 due, at least in part, to potential damage to the spinal cord. For vertebrae above L3 the BVN and spinal canal are too close together. A study of the BVN foramina shows the distance from the nerve root to the edge of the vertebrae decreases by 22% from L3 to L2.

As described herein, a RF (radiofrequency) electrode can be constructed with at least one directional element to bias the ablation toward a particular direction or range of directions. For example, a RF electrode with at least one directional element can be positioned to bias the ablation volume toward the BVN with less ablation in the direction of the spinal cord. Examples of directional elements include directional electrodes, directional fluid delivery ports, or any combination thereof.

A RF ablation system can include a bipolar RF electrode (i.e., a component with two electrodes on the same shaft), instead of two or more monopolar electrodes. In at least some embodiments, a bipolar RF electrodes includes one electrode that supplies power while the other electrode acts as a return. Each electrode of the bipolar RF electrode requires one channel of the RF generator. In at least some embodiments, an RF generator that was previously used for monopolar electrodes can be used or adapted for use with a bipolar RF electrode. The bipolar RF electrode will be used herein for illustrative purposes. It will be recognized that the directional elements and other features described herein can also be applied to monopolar RF electrodes and other multipolar RF electrodes.

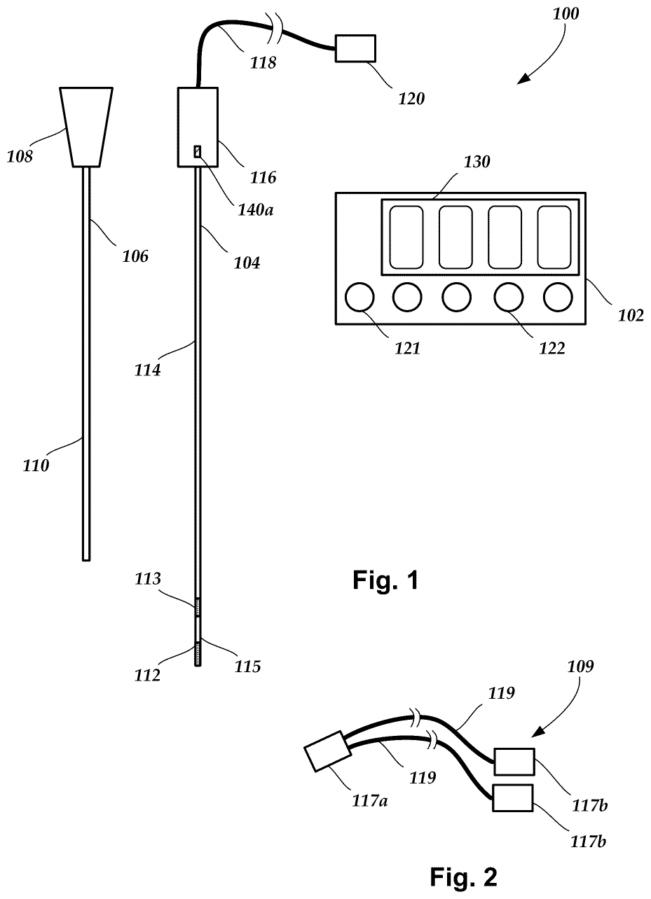

FIG. 1 illustrates one embodiment of a RF ablation system 100 that includes a RF generator 102, a bipolar RF electrode 104, and a cannula 106. It should be appreciated that other RF electrodes may be used in place of the bipolar RF electrode, such as a multipolar RF electrode having multiple electrodes on the same shaft or at least one monopolar RF electrode. A bipolar RF electrode 104 is used herein for illustrative purposes, but it will be understood that the directional elements described herein can be used with monopolar RF electrodes and other multipolar RF electrodes. It will be recognized that some embodiments of a RF ablation system can include more or fewer components, such as, for example, multiple RF electrodes.

The cannula 106 includes a cannula hub 108 and a cannula shaft 110. The cannula shaft 110 is hollow for receiving the bipolar RF electrode 104. The bipolar RF electrode 104 includes an electrode shaft 114, a first electrode 112, a second electrode 113, an insulative material 115 (which may be part of the electrode shaft) separating the first and second electrodes, an electrode hub 116, a cable 118 that is electrically coupled to the electrode shaft 114, and a connector 120 for coupling to at least one port 122 of the RF generator 102 to energize the first electrode 112 or second electrode 113 (or both) via the cable 118 and connector 120.

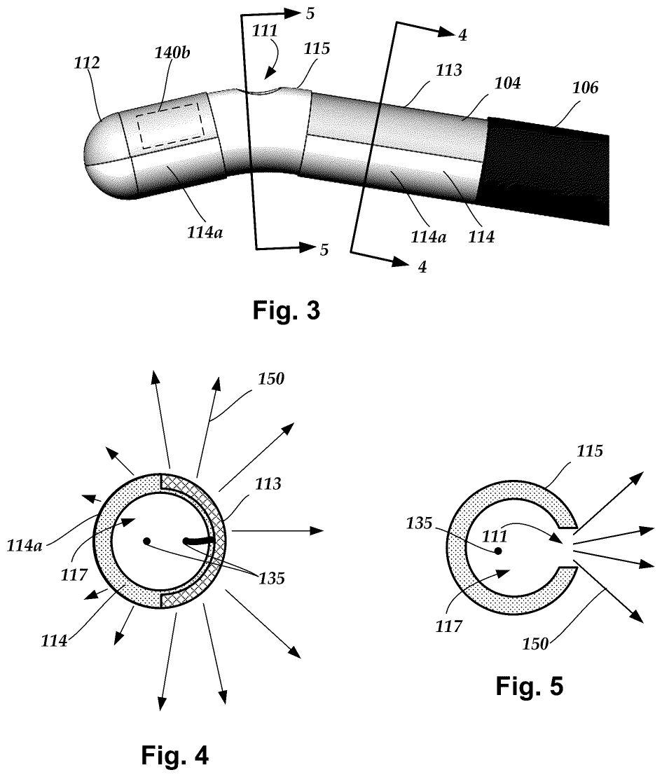

The electrode shaft 114 can be formed using one or more pieces. In at least some embodiments, the insulative material 115 is part of the electrode shaft 114. In at least some embodiments, the first and second electrodes 112, 113 are coupled to, or disposed along, one end portion of the electrode shaft 114 with the electrode hub coupled to, or disposed on, the opposite end portion of the electrode shaft. For example, the first and second electrodes 112, 113 can be attached, or disposed on, one end portion of the electrode shaft 114, as illustrated in FIGS. 1 and 3. In at least some embodiments, the electrodes 112, 113 are attached to the electrode shaft 114 using any suitable method including, but not limited to, adhesive attachment, attachment by reflow the material of the electrode shaft, attachment by injection molding to form at least a portion of the electrode shaft, or the like or any combination thereof.

The RF generator 102 can include one or more ports 122 and at least one screen 130. In at least some embodiments, each port 122 is associated with a portion of the screen 130 (or a different screen) and can receive the connector 120 from a bipolar RF electrode 104 or a connector from an adapter 109 (FIG. 2), as described below. Information such as current, voltage, impedance, status, or the like or any combination thereof can be displayed on the screen 130. In at least some embodiments, each port 122 corresponds to an independent channel. The RF generator 102 optionally includes a ground port 121.

Examples of RF generators and RF ablation systems and methods of making and using the RF generators and RF ablation systems can be found at, for example, U.S. Pat. Nos. 9,717,552; 9,956,032; 10,111,703; 10,136,937; 10,136,942; 10,136,943; 10,194,971; 10,342,606; 10,363,063; 10,588,687; 10,631,915; 10,639,098; and 10,639,101; U.S. patent Application Publications Nos. 2014/0066917; 2014/081260; 2014/0121658; 2021/0121224; 2021/0236191; 2022/0202484; 2022/0202485; and 2022/0226039; U.S. patents applications Ser. No. 17/553,555 and Ser. No. 17/574,400; and U.S. Provisional Patent Applications Ser. Nos. 63/413,122 and 63/413,133, all of which are incorporated herein by reference in their entireties. At least some of these reference include examples of bipolar RF electrodes that can be modified to include at least one directional element, as described herein, as well as systems and methods that utilize the bipolar RF electrodes described herein.

The bipolar RF electrode 104 has two conductors 135 (FIGS. 4 and 5), for example, wires, that extend along the cable 118, optionally through the electrode shaft 114, and couple to the first and second electrodes 112, 113, respectively. In at least some embodiments, one conductor 135 is electrically coupled to one of the electrodes (for example, electrode 112) and supplies power to that electrode and the other conductor 135 is electrically coupled to the other one of the electrodes (for example, electrode 113) and acts as a return. In at least some embodiments, at least the electrode shaft 114, the second electrode 113, and the insulative material 115 have a hollow interior 117 (FIGS. 4 and 5) to allow passage of the conductors 135, and, optionally, fluid, through the bipolar RF electrode 104.

In at least some embodiments, the conductors 135 (FIGS. 4 and 5) are insulated. In at least some embodiments, at least one (or both) of the first and second electrodes 112, 113 are insulated within the hollow interior 117 (FIGS. 4 and 5) defined by at least the electrode shaft 114, the second electrode 113, and the insulative material 115. For example, the second electrode 113 can be disposed over a portion of the electrode shaft 114 (for example, a plastic or polymeric electrode shaft) with an opening in the electrode shaft for passage of a conductor 135 and connection to the second electrode, as illustrated in FIG. 4. The first electrode 112 can be inserted into the tip of the electrode shaft 114 or insulative material 115. (In at least some embodiments, the insulative material 115 is part of the electrode shaft 114.) Insulation of the conductors 135 and at least one of the first or second electrodes 112, 113 may reduce or prevent shorting of the first and second electrodes due to fluid residing in, or flowing through, the bipolar RF electrode 104.

At least some RF generators provide a single channel at each port 122. In at least some embodiments, the bipolar RF electrode 104 uses a separate channel for each of the two electrodes 112, 113. In at least some embodiments, the RF ablation system 100 can include an adapter 109, illustrated in FIG. 2, with a connector 117a to connect to the connector 120 of the bipolar RF electrode 104, two cables 119 that are individually coupled through the connector 117a to a different one of the conductors 135 (FIGS. 4 and 5) of the bipolar RF electrode, and two port connectors 117b for coupling to individual ports 122 of the RF generator 102. This permits one port 122 to energize one of the electrodes (for example, electrode 112) and another port 122 to act as a return using the other of the electrodes (for example, electrode 113.)

FIG. 3 is a close-up view of distal end portions of one embodiment of the bipolar RF electrode 104 and cannula 106 with the first electrode 112, second electrode 113, and, optionally, at least one fluid delivery port 111 in the insulative material 115 between the first and second electrodes. In FIG. 3, the first and second electrodes 112, 113 are directional electrodes (i.e., directional elements) that only partially extend around the circumference of the electrode shaft 114. In the illustrated embodiment of FIG. 3, the first and second electrodes 112, 113 extend around only 50% of the circumference of the electrode shaft 114. In at least some embodiments, one or both of the first and second electrodes 112, 113 extend around no more than 20, 30, 40, 50, 60, 70, or 80 percent of the circumference of the electrode shaft 114. The exposed remainder 114a of the electrode shaft 114, opposite the first or second electrode 112, 113, is not conductive.

During ablation using at least one direction electrode, the ablation volume will typically be biased in the directions away from the bipolar RF electrode that correspond to the portion of the circumference of the electrode shaft 114 around which the directional electrode(s) (for example, the first and second electrodes 112, 113 of FIG. 3) extend(s). Thus, the ablative effect is expected to extend further in the directions away from the bipolar RF electrode that correspond to the portion of the circumference of the electrode shaft 114 around which the directional electrode(s) (the first and second electrodes 112, 113) extend(s). The ablative effect is expected to extend less in directions away from the regions of bipolar RF electrode where there is no portion of the first or second electrodes 112, 113. This difference in the extent of the ablative effect into the tissue is represented by the length of the arrows 150 in the cross-sectional view of FIG. 4.

In at least some embodiments, both the first and second electrodes 112, 113 are directional electrodes, as illustrated in FIG. 3. In at least some embodiments, the first and second electrodes 112, 113 are directional electrodes that are aligned with each other on the circumference of the electrode shaft with the first and second electrodes extending around the same portion of the circumference of the electrode shaft 114 (although at different longitudinal positions along the length of the electrode shaft). In at least some embodiments, the first and second electrodes 112, 113 are directional electrodes that extend around different portions of the circumference of the electrode shaft 114 (and at different longitudinal positions along the length of the electrode shaft). These different portions of the circumference of the electrode shaft 114 may or may not circumferentially overlap (although at different longitudinal positions along the length of the electrode shaft).

In at least some embodiments, only one of the first and second electrodes 112, 113 is a directional electrode and the other one of the first and second electrodes is a non-directional electrode that extends around the entire circumference of the electrode shaft 114. In at least some of these embodiments, the electrode that is to be energized is a directional electrode and the return electrode is a non-directional electrode.

In at least some embodiments, the bipolar RF electrode 104 can include a marker for identification of the circumferential orientation or position of one or more directional elements, such as one or more directional electrodes or one or more directional fluid flow ports (described in more detail below) or any combination thereof. One example is a marker 140a positioned on the electrode hub 116 (or electrode shaft 114), as illustrated in FIG. 1. The marker 140a is placed so that the marker is circumferentially aligned with the directional electrode(s) (for example, first and second electrodes 112, 113 of FIG. 3) or directional fluid flow ports (for example, fluid flow port 111 of FIG. 3). Observation of the marker 140a by a user will identify the circumferential orientation of the directional elements on the distal portion of the bipolar RF electrode 104.

Another example of a marker is the marker 140b of FIG. 3. The marker 140b is made of a radiopaque material, such as stainless steel, and is positioned relative to the directional element(s) (such as the first electrode 112 or the fluid flow port 111 of FIG. 3) so that radiological observation of the marker 140b (for example, by fluoroscopy) will indicate the circumferential orientation or position of the directional element(s). For example, in FIG. 3, the marker 140b is positioned with the electrode shaft 114 beneath the first electrode 112 and distinguishes that portion of the electrode shaft that is covered by the first electrode 112 from the remainder 114a of the electrode shaft. It will be understood, that the marker 140b could be positioned under the second electrode 113 or elsewhere within the electrode shaft 114 or insulative material 115 with the placement identifying the circumferential orientation or position of at least one of the directional element(s) of the bipolar RF electrode 104.

In at least some embodiments, as illustrated in FIG. 3, the distal end of the bipolar RF electrode 104 can include a bend 115a. In the illustrated embodiment and at least some other embodiments, the bend 115a is formed in the insulative material 115 between the first and second electrodes 112, 113. In other embodiments, alternatively or additionally one (or both) of the electrodes 112, 113 include(s) a bend. The bend 115a can facilitate placement of the electrodes 112, 113 near the BVN in the vertebra.

In at least some embodiments, the first electrode 112 is a tip electrode that is not open at the distal end, as illustrated in FIG. 3. In at least some embodiments, the first electrode 112 is a tip electrode that is capped or closed at the distal end.

A bipolar RF electrode (or other RF electrode) can be constructed to allow for fluid flow between the two electrodes (in the case of a bipolar RF electrode), or otherwise near the electrode(s), for delivery of fluid, drugs, medications, contrast agents, or the like through the bipolar RF electrode and directly to the ablation site. In contrast, fluid or drugs delivered through the cannula used to insert the bipolar RF electrode would likely be delivered at a site that is a significant distance (for example, 15 to 40 mm or more) away from the ablation site. The response or effect of the fluid delivery through the cannula to the ablation site can be inconsistent or unpredictable due to this distance.

In at least some embodiments, as illustrated in FIG. 3, the distal end of the bipolar RF electrode 104 can include at least one fluid delivery port 111 defined in the insulative material or electrode shaft and disposed between the first and second electrodes. In other RF electrode configurations, the fluid delivery port(s) 111 can be placed in other arrangements relative to the electrode(s).

A fluid delivery port 111 can be a directional element with the fluid flow being directed away from the fluid delivery port 111. Directional fluid delivery can be performed using one or more fluid delivery ports 111 that are not uniformly distributed around the circumference of the bipolar RF electrode 104. Any suitable number of fluid delivery ports 111 can be used including, but not limited to, one, two, three, four, or more fluid ports. In at least some embodiments, the bipolar RF electrode 104 includes a single fluid delivery port 111 that, at its widest, extends around no more than 20, 30, 40, or 50 percent of the circumference of the electrode shaft. In at least some embodiments, the bipolar RF electrode 104 includes at least two fluid delivery ports 111 that are defined in a contiguous portion of the electrode shaft that extends around no more than 20, 30, 40, or 50 percent of the circumference of the electrode shaft.

The placement and size of the fluid delivery port(s) 111 determines the direction(s) of flow of fluid from the bipolar RF electrode 104. The embodiment illustrated in FIG. 3 has a single fluid delivery port 111 with fluid flow represented by the arrows 150 in the cross-sectional view of FIG. 5. In the illustrated embodiment of FIG. 3, the flow of fluid will be primarily out of the fluid flow delivery port 111 in the same or similar direction(s) as the flow of RF energy from the first and second electrodes 112, 113.

Additional examples and descriptions of bipolar RF electrodes having at least one fluid delivery port are presented in U.S. Provisional Patent Application 63/440,612, filed on Jan. 23, 2023, incorporated herein by reference in its entirety.

A bipolar RF electrode 104 can include one or more directional electrodes with or without any fluid delivery ports 111. A bipolar RF electrode 104 can include one or more fluid delivery ports 111 for directional fluid flow with or without directional electrode(s). A bipolar RF electrode 104 can include any combination of one or more directional electrodes and one or more fluid delivery ports 111 for directional fluid flow. In the illustrated embodiment of FIG. 3, the bipolar RF electrode 104 includes two directional electrodes (first and second electrodes 112, 113) and a single fluid delivery port 111.

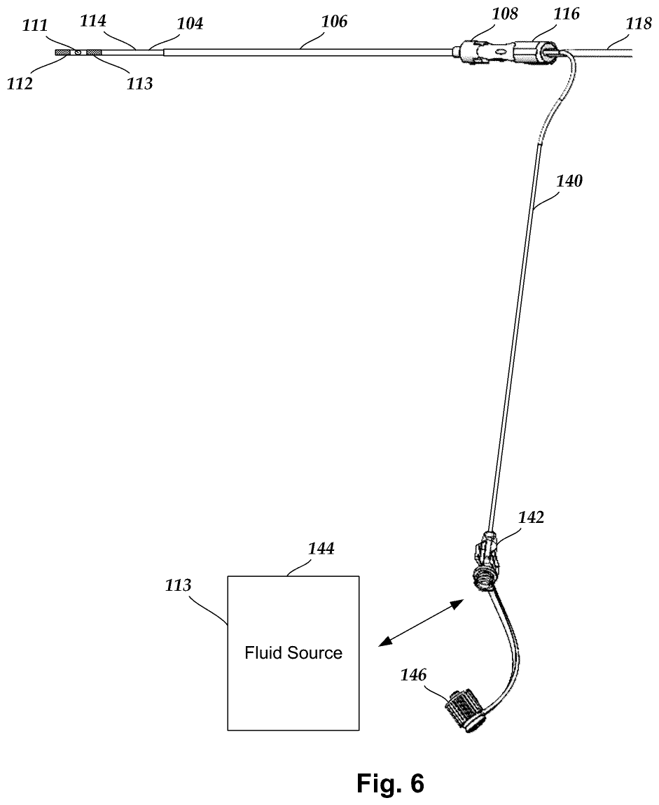

FIG. 6 illustrates one embodiment of the bipolar RF electrode 104 disposed in the cannula 106 and a fluid line 140 extending from the electrode hub 116 or electrode shaft 114 of the bipolar RF electrode. In at least some embodiments, the fluid line 140 is a flexible tubing. The fluid line 140 is in fluid communication with the interior 117 (FIGS. 4 and 5) defined by at least the electrode shaft 114, the second electrode 113, and the insulative material 115. The fluid line 140 is attached to a connector 142, such as a Luer connector, that can be coupled to a fluid source 144, for example a syringe, for delivery of fluids through the fluid line 140, electrode shaft 114, and out the at least one fluid delivery port 111 between the first and second electrodes 112, 113. The connector 142 optionally includes a cap 146.

In at least some embodiments, an electrically conductive fluid, such as water, saline, or any other conductive fluid or fluid that becomes conductive when mixed with bodily fluids at the target site, is delivered through the at least one fluid delivery port 111 to increase the conductivity at the ablation site and to ensure that the desired target nerve, such as the BVN, or other tissue is ablated. This can result in a larger ablation volume than if no fluid was delivered because RF energy needs a conductive medium to increase the temperature to ablate the tissue.

When the fluid delivery port(s) 111 are directional elements, the ablation volume can be biased in the direction of fluid flow from the fluid delivery port(s). As an example, the first and second electrodes of the bipolar RF electrode can be inserted in the vertebra in a position between the BVN and the spinal cord. The fluid delivery port(s) are aligned toward the BVN so that the ablation volume is biased toward the BVN with less of the ablation volume toward the spinal cord. In at least some embodiments, at least one of the first and second electrodes 112, 113 is a directional electrode and the fluid delivery port(s) 111 is/are a directional element and the fluid delivery port(s) 111 is/are directionally aligned with the directional electrode(s) with the fluid delivery port(s) arranged over the same portion of the circumference of the electrode shaft 114 (although at different longitudinal positions along the length of the electrode shaft) over which the directional electrode(s) extend. FIG. 3 illustrates one example of a single fluid delivery port 111 directionally aligned with two directional electrodes (the first and second electrodes 112, 113).

As another example of use, before ablation a clinician can deliver a numbing agent, such as lidocaine, through the at least one fluid delivery port 111 between the first and second electrodes 112, 113. A significant decrease in the patient's pain confirms that the bipolar RF electrode 104 is in the correct position to ablate the desired nerve, such as the BVN, or other tissue. The numbing agent can also relieve or reduce any discomfort or pain felt by the patient during ablation. Moreover, in at least some embodiments, the use of the numbing agent can confirm that the patient is a good candidate for ablation therapy.

Ablation, such as BVN ablation, does cause harm to the target tissue, such as the BVN. The unablated nerve endings subsequently heal or repair themselves. In a least some embodiments, the clinician can inject healing medication, such as a steroid, through the at least one fluid delivery port 111 between the first and second electrodes 112, 113 to speed up the healing process. Faster healing may reduce the painful time interval after the ablation procedure.

Another use for the fluid port can be to inject a contrast agent through the at least one fluid delivery port 111 between the first and second electrodes 112, 113 to facilitate imaging and visualization of the target site using, for example, MRI, fluoroscopy, or the like or any combination thereof.

In at least some embodiments, embolic beads can be passed through the at least one fluid delivery port 111 between the first and second electrodes 112, 113 to starve the nerves or other target tissue of blood flow and effectively kill the nerve or other tissue, such as a tumor, as an alternative or supplement to ablation.

The above specification provides a description of the structure, manufacture, and use of the invention. Since many embodiments of the invention can be made without departing from the spirit and scope of the invention, the invention also resides in the claims hereinafter appended.

What is claimed as new and desired to be protected by Letters Patent of the United States is:

1. A bipolar RF electrode, comprising:
an electrode shaft having a circumference, a first end portion, and a second end portion opposite the first end portion;
a first electrode coupled to the second end portion of the electrode shaft, wherein the first electrode extends around no more than 80% of the circumference of the electrode shaft;
a second electrode coupled to the second end portion of the electrode shaft;
an insulative material coupled to, and disposed between, the first electrode and the second electrode; and
an electrode hub attached to the first end portion of the electrode shaft.

2. The bipolar RF electrode of claim 1, wherein the second electrode extends around no more than 80% of the circumference of the electrode shaft.

3. The bipolar RF electrode of claim 2, wherein both the first electrode and the second electrode extend around no more than 50% of the circumference of the electrode shaft.

4. The bipolar RF electrode of claim 3, wherein the first electrode and the second electrode are aligned on the circumference of the electrode shaft.

5. The bipolar RF electrode of claim 1, wherein the insulative material defines at least one fluid delivery port.

6. The bipolar RF electrode of claim 5, wherein the electrode hub or the electrode shaft is configured for attachment of a fluid line, wherein at least the electrode shaft, the second electrode, and the insulative material form a hollow interior for flow of fluid from the electrode hub or the electrode shaft to the at least one fluid delivery port defined by the insulative material and disposed between the first electrode and the second electrode.

7. The bipolar RF electrode of claim 5, wherein the at least one fluid delivery port is a single fluid delivery port directionally aligned with the first electrode.

8. A kit, comprising:
the bipolar RF electrode of claim 1; and
a cannula configured for insertion of the electrode shaft through the cannula.

9. The kit of claim 8, further comprising the fluid line coupled, or coupleable, to the electrode shaft or the electrode hub.

10. A RF ablation system, comprising:
the bipolar RF electrode of claim 1;
a cannula configured for insertion of the electrode shaft through the cannula; and
a RF generator configured for electrically coupling to the bipolar RF electrode and energizing at least one of the first electrode or the second electrode.

11. A bipolar RF electrode, comprising:
an electrode shaft having a first end portion and a second end portion opposite the first end portion;
a first electrode coupled to the second end portion of the electrode shaft;
a second electrode coupled to the second end portion of the electrode shaft;
an insulative material coupled to, and disposed between, the first electrode and the second electrode, the insulative material having a circumference and defining one or more fluid delivery ports, wherein, when the one or more fluid delivery ports is a single fluid delivery port, the single fluid delivery port extends around no more than 50% of the circumference of the insulative material and, when the one or more fluid delivery ports is at least two fluid delivery ports, all of the at least two fluid delivery ports are defined in a contiguous portion of the insulative material that extends around no more than 50% of the circumference of the insulative material; and
an electrode hub attached to the first end portion of the electrode shaft, wherein the electrode hub or the electrode shaft is configured for attachment of a fluid line, wherein at least the electrode shaft, the second electrode, and the insulative material form a hollow interior for flow of fluid from the electrode hub or the electrode shaft to the at least one fluid delivery port defined by the insulative material and disposed between the first electrode and the second electrode.

12. The bipolar RF electrode of claim 11, wherein the first electrode extends around no more than 80% of the circumference of the electrode shaft.

13. The bipolar RF electrode of claim 11, wherein both the first electrode and the second electrode extend around no more than 50% of the circumference of the electrode shaft.

14. A RF ablation system, comprising:
the bipolar RF electrode of claim 11;
a cannula configured for insertion of the electrode shaft through the cannula; and
a RF generator configured for electrically coupling to the bipolar RF electrode and energizing at least one of the first electrode or the second electrode.

15. A RF electrode, comprising:
an electrode shaft having a circumference, a first end portion, and a second end portion opposite the first end portion;
at least one electrode coupled to the second end portion of the electrode shaft, wherein the at least one electrode comprises a first electrode that extends around no more than 80% of the circumference of the electrode shaft; and
an electrode hub attached to the first end portion of the electrode shaft.

16. The RF electrode of claim 15, wherein the first electrode extends around no more than 50% of the circumference of the electrode shaft.

17. The RF electrode of claim 15, wherein the electrode shaft defines at least one fluid delivery port.

18. The RF electrode of claim 17, wherein the electrode hub or the electrode shaft is configured for attachment of a fluid line, wherein at least the electrode shaft forms a hollow interior for flow of fluid from the electrode hub or electrode shaft to the at least one fluid delivery port defined by the electrode shaft.

19. The RF electrode of claim 17, wherein the at least one fluid delivery port is a single fluid delivery port directionally aligned with the first electrode.

20. A RF ablation system, comprising:
the RF electrode of claim 15;
a cannula configured for insertion of the electrode shaft through the cannula; and
a RF generator configured for electrically coupling to the RF electrode and energizing the at least one of the at least one electrode.
